(19) United States
(12) Patent Application Publication
(10) Pub. No.: US 2010/0021512 A1
(43) Pub. Date: Jan. 28, 2010

(54) Incontinence garments with a silver lining infection stopper

(76) Inventor: Stanley Arron, BridgePort, CT (US)

Correspondence Address: Stanley Arron, 3323 Old Town Road, Bridge Port, CT, 06606, US

(21) Appl. No.: 12/220,355

(22) Filed: Jul. 24, 2008

(51) Int. Cl. A61L 15/18; A61K 33/38; A61P 31/00

(52) U.S. Cl. 424/404; 424/618

(57) ABSTRACT

Antibiotics used to cure Uterine Tract Infections have resulted in the growth of MRSA. and other antibiotic resistant bacteria. The CDC guidelines state “Disposable diapers cause 95% of all UTI'S”. The procedures used for “prevention” are ineffective because millions of bacteria in incontinence garments continue to double every 20 minutes. Silver ions in bandages are used to cure severly damaged skin, such as in burns. The best two in a large scale test achieved results in just 30 minutes. This Invention uses silver ions in linings next to the skin to stop the growth of over 250 kinds of infectious bacteria on it's surface and the body parts it touches. It achieves results in 30 minutes, equal to the most highly rated bandages . . . for every level of incontinence for both sexes.

### BACKGROUND OF THE INVENTION

We have all been told that after “toileting” we should wash our hands thoroughly without our knowing how much “infectious” material there is in our underwear. The work of Charles Gerba at the University of Arizona in the latter part of the 1900's disclosed enough contamination from underwear in the average wash to contaminate the entire load and the machine. Females suffer most from Urinary Tract Infections (UTI'S) because their Urinary Orifices are more accessible to infectious bacteria. UTI'S are common and cured by Antibiotics. Over the years, bacteria developed immunities to Antibiotics. The most serious is MRSA (Methacillin Resistant Staphaloccus Aureus}.

The most affected are the elderly and Impaired in Long Term institutions and Hospitals where disposable diapers and briefs are used on many residents. The Center for Disease Control Guidelines state that 95% of the UTI'S are caused by disposable diapers. Cured by Antibiotics, 20% of women get recurrences, 20% of this group get a second infection and are in danger of developing their personal Antibiotic resistant strain of bacteria. Consequently, in the event of another illness, they may be difficulty in finding an Antibiotic that can cure them.

APIC (THE ASSOCIATION FOR INJECTION CONTROL AND EPIDEMIOLOGY) states that each year, 1.7 million people contact infections at a healthcare facility. Each year, more than 99,000 people die due to HEALTH CARE ASSOCIATED INFECTIONS (HAI). They state “PREVENTION IS A SOLUTION”. This is misleading because it does not refer to “Prevention from creation of the infection”. They are concerned with the “spreading” of the infection. They talk about washing hands, cleaning instruments, care of catheters, use of antibiotics, etc.

This invention is concerned with preventing creation of the infection. The hot, damp, inside surface of disposable diapers, pads and pants promote bacterial growth.

An elderly female sitting in a wheelchair for 5 hours has about 24 million bacteria on the disposable pressing inward toward her Urinary Orifice. This invention places a “Silver Lining” barrier on the surface of the disposable to keep bacteria from growing and moving onto the body of the patient.

### PRIOR ART

U.S. Pat. No. 4,695,279 for incontinence briefs and pants. Is based on a pocket of liquid impermeable materials built Into the crotch with an open section to hold a disposable pad. The additional heat created by the impermeable material helps to promote growth of infection bacteria on the surface of the pad, which is next to the skin, and a female's urethral orifice.

British Patent No 1,178,212 discloses a lining of waterproof material in the crotch forming a front and back pocket for the purpose of holding an absorption pad such as a sanitary napkin. Welded seams form a gap to hold the end of the absorbent pad. There is no provision for preventing bacterial growth on it's surface, next to a female's urethral oriface.

British Patent No 1,441,087 (Kanga) shows a pocket open at one end to hold a removable pad of absorbent material. It grows infectious bacteria next to a female's urethral oriface.

British Patent No 1,580,550 is for a panty with a holder for a sanitary napkin. The holder is of waterproof material for insertion of a sanitary napkin. Closure of the flaps is with “Velcro” which provides no liquid barrier under the closure. Odor causing and infectious bacteria thrive near the orethral oriface.

International Patent No WO85/03430 (Brier) A disposable waterproof holder for an absorbent sanitary pad is releasably fixed to an undergarment to convert it to a sanitary or incontinence garment. Infectious bacteria grows on it's surface.

### SUMMARY OF PRIOR ART

The most commonly voiced complaint about “Disposable” diapers, pants and pads was that they are bad for the because they could take centuries to disintegrate in landfills. The facts are and that these garments grow bacteria contributing to 99,000 deaths yearly, billions of $ yearly to cure infections, and constitute a crisis. The prior art shows Incontinence garments that double the amount of infectious bacteria on their surfaces every 20 minutes. They are a major source of Uterine Tract Infections. Curing them with Antibiotics created MRSA.

### OBJECTS, AND ADVANTAGES OF THIS INVENTION

This invention prevents suffering by patients, lessens Infection exposure to caregivers and costs to institutions. I have a simplified structure of an Anti microbial lining in the crotch, a bottom of liquid impervious fabric, and a tunnel for the easy insertion of an absorption means between the parts.

The silver ions in the lining stop the growth of over 250 kinds of infectious microbes on it's surface and the skin. My personal experience is that I allowed urine and bacteria in my personal incontinent briefs to grow & infect me. After changing to a pants with a Silver lining in the crotch, the itching created disappeared in 30 minutes. Silver ions in bandages kill bacteria in wounds in 30 minutes. We duplicate that on intact skin.

Costs of use are reduced dramatically. For minor leaks and drips, folded sheets of paper towels absorb urine instantly, retain strength when wet and can be flushed away in the toilet. For moderate losses, there are absorbents that can be flushed or disposed in the garbage. Reusable Needle Punch Polyesters can often be laundered 100 or more times.

Traditional disposable pads can also be worn under the Silver Lining thereby keeping a clean surface next to the body.

The body parts of the briefs can incorporate Anti Microbial silver for enhanced protection of care givers in long term facilities and hospitals. There is very little, or no evidence, that Silver allows bacteria to mutate into resistant strains. It attacks the cells vital structure, including the cell wall and RNA function.

Disposable pants and diapers are a special problem. Each one is a complete assembly of 1) an inside acquisition layer next to the body 2) a waterproof bottom and 3) an absorption means between them. They are worn by the most susceptible elderly females. My discovery is that “Velcro” type hooks will also close onto the inside surface of the disposable diapers and pull on pants. Two or more “Velcro” spaced hook parts sewn to the bottom press into the disposable and create a “clean top”.

### BRIEF DESCRIPTION OF THE DRAWINGS

FIG. 1 Is a top side view of the liquid impervious base of the crotch assembly. At one end it will be joined to the bottom of the matching front body part. The other end will be joined to the matching back body part. The size illustrated can be enlarged or reduced to conform to the design of particular garments,

FIG. 2 Is a top view of an Anti-Microbial lining of a size and shape that fits in the crotch above the liquid impervious bottom. of FIG. 1 It is centered and joined to this part along each of it's long length sides; leaving an opening at the front and back ends. This creates a tunnel into which an absorption means can be inserted. This structure does not need the extra material and labor costs of making a pocket to hold an absorbent or disposable pad.

FIG. 3 Is a top view of an absorption means that can be inserted and removed from either the front or back end of the tunnel. The absorption means in this construction does not need an acquisition top or liquid impervious bottom, which is a labor and material saving. The absorption means can be flushable, disposable or reusable.

FIG. 4 Is a front top view of the open parts showing the back body part, the front body part, the anti-microbial crotch lining assembly in the center and the “Velcro-press to close” parts.

FIG. 5 Is a front top view of the body parts and Velcro parts in closed condition. The Velcro “hook” parts on the inside edges of the back side extensions can close to the Velcro “loop” parts anywhere along the “loop”, thereby allowing the garment to be worn by a multiple of body sizes.

FIG. 6 Is an exploded top front open view to better show the position of the anti-microbial lining in the crotch, or perineal area. The body parts can be a diaper, side opening or pull on style as long as the integrity of the crotch assembly position is maintained

FIG. 7 A rectangular part of Anti Microbial fabric with overlock or hemmed stitched edges around it's perimeter.

FIG. 8 Two parts of Velcro Hooks that can be pressed into the Inside body parts of disposable diapers or pull on pants to make a releasable closure.

FIG. 9 The Velcro hook parts of FIG. 8 are sewn into position near each end of the anti microbial fabric of FIG. 7.

FIG. 10 A top view of a disposable pad Part 123

### DETAILED DESCRIPTION OF THIS EMBODIMENT

This invention presents a way to control the spread of Multi-drug Resistant Organisms in underwear, with emphasis on Incontinence garments. The need for this is expressed by an editorial in the Mar. 12, 2008 issue of the Journal of the American Medical Association. The subject is a report by Drs Daniel J. Diekema and Michael Climo, titled “PREVENTING MRSA (METHICILLIN-RESISTANT STAPHYLOCOCCUS INFECTIONS): FINDING IT IS NOT ENOUGH”. Their theme is “more is needed” to stop spread of the infections. This invention stops “creation”.

These parts are sewn together with standard commercial sewing machines available everywhere. The central assembly covers the perineal (crotch} area. The body parts and the crotch sides have elastic components that hold a silver lining in close contact with the body of the wearer. Bandages containing silver ions are very effective in serious wound care. We have created a “bandage like” effect that prevents the growth of over 250 kinds of microbes on the surface of the lining and the body parts it touches. These features are also incorporated into pull on pants.

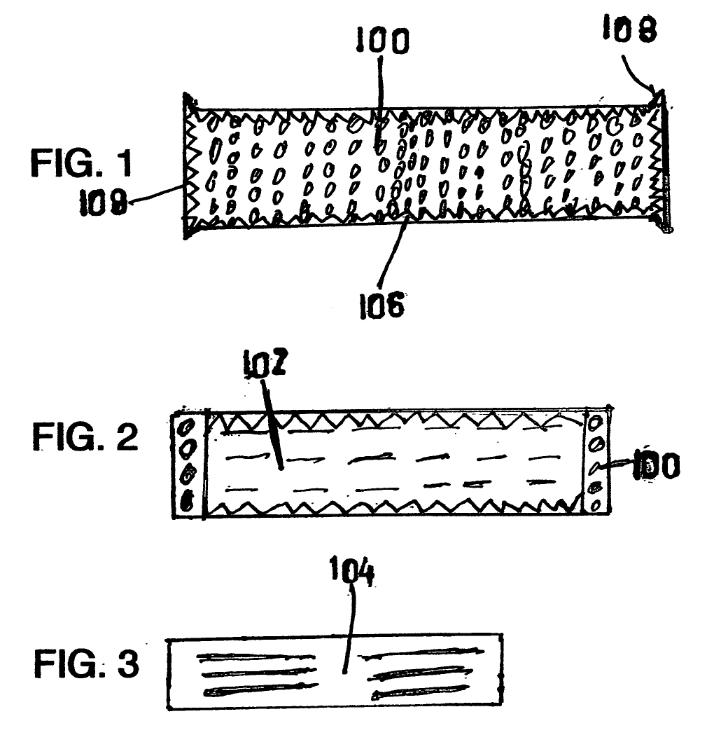

Referring to FIG. 1. Part 100 is a liquid impermeable bottom of the crotch part; fitting between the front and rear body parts. The fabric is a coated nylon taffeta. Purchased from First Performance Fabrics, of New York, N.Y.

Referring to FIG. 2. Part 102 Is a 100% Polyester Anti-microbial lining that fits next to the skin. This fabric contains Silver Ions which are activated by moisture to stop the growth of over 250 kinds of microbes on it's surface. This fabric is held in close contact to the skin by elastic in the body parts and the elastic leg bindings in the crotch. This creates a “bandage” effect. “Silver” in bandages is used to heal the most serious wounds, such as burns. The Silver lining is skin friendly and has a parallel prevention function.

The Silver Lining fabric is Style 692430, purchased from Milliken & Company, 1045 Sixth Avenue, New York, N.Y.

The silver lining part 102 is centered above part 100 and joined to it by sewing stitches 106 only along their two long sides. The front and back edges are left open. This creates a tunnel into which a variety of absorption means can be inserted.

Referring to FIG. 3. Part #104 represents an absorption means that can be inserted into the tunnel and removed after being used. Disposable absorption pads usually are composed of three parts. 1. A top acquisition layer 2. A liquid Impermeable bottom and 3. an absorption means between the two. Our construction just disclosed features an enhanced wicking layer containing silver next to the skin and a liquid impermeable bottom. All that is needed is an absorption means. The labor and fabric costs of disposable pads that have to be disposed in landfills are saved. Part 106 Shows the sewing joining the long sides of part 102 to Part 100. Part 108 shows the sewing stitches joining the front and rear body parts to the liquid impervious center.

For low level “stress” leaks and drips, the absorption means can be as low cost as paper towels that maintain their strength when wet, and can be disposed in the garbage. Flushable absorbents and reusable needle punched fabrics can be used for larger losses.

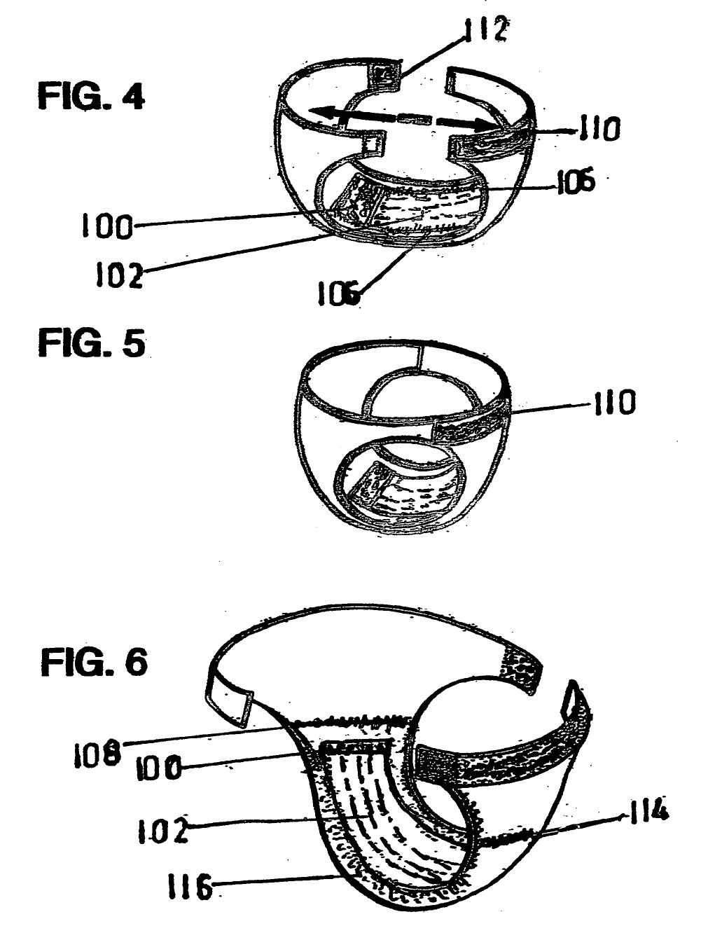

Referring to FIG. 4 This shows the side opening garment with the top open. Velcro closure parts loop part #110 and hook parts #112. The #102 anti microbial silver lining is in place in the crotch.

Referring to FIG. 5 This shows the side opening garment of FIG. 4 with the velcro parts in closed position.

Referring to FIG. 6 This is an exploded view of the side opening brief to show position of the #102 Anti microbial silver lining in the crotch. Stitching #114 shows where the liquid impermeable part is joined to the front and rear body parts. #116 shows the edge elastic on both sides of the centered silver lining and liquid impermeable fabric. The edge elastic #116 holds the skin friendly silver anti microbial lining close to the wearer's body parts.

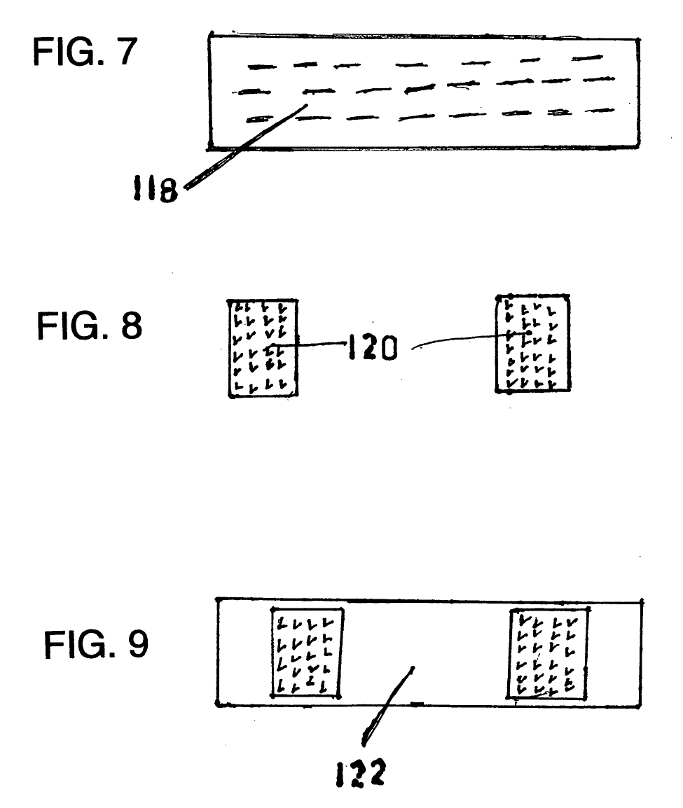

Referring to FIGS. 7, 8 and 9. These show a solution to the problem of the growth of infectious bacteria on the inside surface of disposable diapers and pants. The magnitude of this problem is in the guidelines of the Center for Disease Control, which state that 95% of the Urinary Tract Infections are caused by disposable diapers. Females are most at risk because the infectious bacteria double in quantity every 20 minutes. After 5 hours, she has over 20 million bacteria pressing up against her urinary Oriface. This invention puts a reusable silver lining between the surface of the disposable and the body. The lining passes the wetness to the absorption part of the disposable, stops the growth of bacteria, and puts a clean surface next her urinary orifice.

FIG. 7 shows #118 which is the same as #102 in FIG. 2.

FIG. 8 shows two hook parts of hook and loop closures. They close onto the insides of disposable garments just as they would grip onto the matching loops. The exact size is not important as the part 118 is very light.

FIG. 9 shows the two #120 hooks sewn near each end of silver lining part #118 with the hook parts on the outside. It is pressed into place in the same position as shown on FIGS. 4, 5, & 6. Reusable. It can be washed with the users personal laundry, where it will also act to disinfect the water.

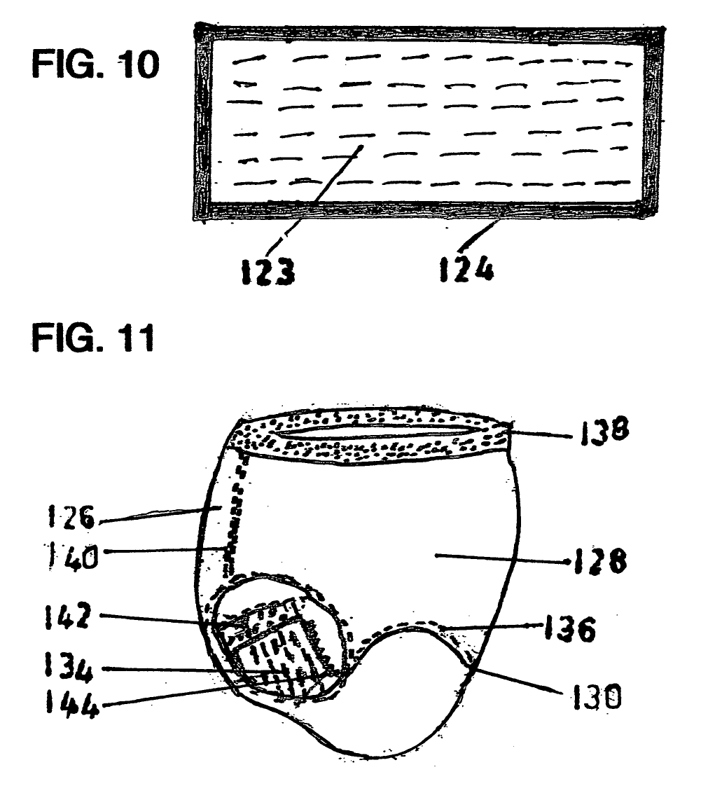

FIG. 10 Is a top view of a disposable pad. Part 123 is the absorption part next to the body. Bottom part 124 is a liquid impervious bottom. It is common for urine to overflow the four sides where part 123 meets part 124. When worn inside my “tunnel”, the high sides of the liquid impermeable part FIG. 1, act to contain side leakage.

FIG. 11 Is a side view of a pair of pull on pants. The body parts, back 126 and front 128 are a stretch knit polyester with “Silver” ions in it's structure; so that it will cling closely to the skin. #140 shows how sewing stitching joins the front to the back, at the sides. #130 shows how the crotch is sewn to the front body part. #132 shows the liquid impervious fabric and the opening into the tunnel formed when it is sewn to “Silver” lining fabric #134 along their long side edges; with stitching #144. #136 stitching of front body part to front of crotch parts. #138 is an elastic waist band sewn to perimeter of top. #140 stitching joins back body part #126 to front body part #128. The same is done on the opposite side. The body fabric can be used to make many men's and women styles with the same crotch structure.

### CONCLUSIONS, RAMIFICATIONS AND SCOPE OF THE INVENTION

DOES IT DO WHAT IT CLAIMS TO DO? The medical community is always skeptical of new advances. They want to see a large scale study proving the new product or procedure does what it is supposed to do. Fortunately, I HAVE SUCH A STUDY. It is in the—Journal of Medical Microbiology (2006),55,59-63.

TITLE—Antimicrobial activities of silver dressings:

an in vitro comparison.

PURPOSE OF PROJECT—Establish anti microbial efficiencies of silver coated or impregnated dressings commercially available.

The Silver coated or impregnated dressings and my linings both use Silver to kill bacteria on the skin and prevent growth of new infections. The best dressings achieved the most rapid killings in 30 minutes, which is what my lining did. All Silver impregnated dressings exerted bactericidal activity. The two best were:

1) ACTICOAT by Smith and Nephew. A polyester & silver coated polyethylene. Killing was rapid with both MRSA & STAPH.
2) CONTREET by Coloplast. A foam dressing with ionic silver. Had maximum killing of MRSA and reduction of bacteria count In 30 minutes.

The “prevention” strategies in use are pathetically Ineffective. These products are needed as soon as possible to reduce the 3 Billion dollars in annual costs and human suffering.

1. Side opening pants that have a shaped anti microbial lining in the crotch, that is longer than it is wide. The lining is joined to a matching shape liquid impervious bottom part. The parts are joined on their matching left and right long sides. They ends are left open to form a tunnel for the purpose of inserting an absorption means between the lining and the liquid impervious bottom.

2. The lining of claim 1, wherein the anti microbial agent in the lining is Silver ions to stop the growth of bacteria on it's surface and the adjoining body parts.

3. The lining of claim 1, wherein liquid impervious fibres wick urine and other body excretions into the lining and pass said body excretions into an absorption means.

4. The pants of claim one, wherein the “Silver” in the lining stops the growth of “MRSA” and other Multi Drug Resistant organisms on it's surface.

5. The pants of claim 1, wherein when the “Silver” lining is held up against the wearers body, the silver stops the growth of infectious bacteria where it touches the body

6. I claim an Anti Microbial fabric with hook parts (of Velcro, or equivalent) that can be releasably pressed into place on the body facing side of disposable diapers and pants; becoming an infection barrier between the disposables and the body.

7. I claim the Anti microbial fabric of claim 6 contains Silver.

8. I claim pull on pants of a stretch fabric with anti microbial silver in it's body parts, a crotch lining containing anti microbial silver, a liquid impervious outer fabric below the lining, and an opening between them for the insertion of an absorption means.

9. I claim the pull on pants of claim 8 with body styles for use by men and women.

10. I claim the pull on pants of claim 8 and the use of a disposable incontinence pad made with an absorption top and a liquid impervious bottom inserted under the anti microbial lining to provide an infection barrier between the top of the disposable pad and the body. We used “Poise” brand pads for this purpose.

11. I claim the pull on pants of claim 8 with narrow flushable, dispersable absorbents shaped to fit in the crotch tunnel to absorb the “stress” urine losses of the wearer. The dispersable absorbent is made by Sellars, 6540 N. Industrial Road, Milwaukee, Wis. 53223.

12. I claim the pull on pants of claim 8 with thin layers of the SELLERS dispersable, flushable, absorbent inserted between the anti microbial lining next to the body and the liquid impervious part under it.

13. I claim the pull on pants of claim 8 with larger size units of dispersable absorbents in the “tunnel” as necessary to hold increased urine losses.
